(19) United States
(12) Patent Application Publication
(10) Pub. No.: US 2006/0025738 A1
(43) Pub. Date: Feb. 2, 2006

(54) Pre-packaged, dispensable diaper with accessories and method of use

(76) Inventors: Cathy D. Santa Cruz, Reno, NV (US); Bonnie Tharp, Reno, NV (US)

Correspondence Address: Cathy D. Santa Cruz, 7630 Tholl Drive, Reno, NV, 89506, US

(21) Appl. No.: 10/899,996

(22) Filed: Jul. 27, 2004

(51) Int. Cl. A61F 13/15

(52) U.S. Cl. 604/385.02; 604/385.13

(57) ABSTRACT

As herein disclosed we provide a pre-packaged dispensable diaper with accessories. The diaper and accessory(s) are packaged and contained within an external wrapping in a sanitary manner. The external wrapping is position able between a folded closed position and an unfolded open position. When the external wrapping is in the unfolded open position it also functions as a sanitary placemat for placement of the baby thereon. Also, the external wrapping is in the form of a disposable bag and thus further functions for disposal of a soiled diaper and any other packages associated with the accessories.

### FIELD OF THE INVENTION

The present invention relates in general to items that may be sold and dispensed from a coin-operated vending machine. Such vending machines are well known and are used for dispensing numerous pre-packaged items. However, the present invention more particularly pertains to a vending machine associated with a baby-changing table such as typically located within most public ladies restrooms. Such baby-changing tables are functional for their intended use but they do not provide any accessories or products that may be useful while changing the baby. Thus, the present invention substantially provides a pre-packaged dispensable diaper changing means for use with a baby-changing table, or the like.

### BACKGROUND OF THE INVENTION

In today's society most public ladies restrooms provide a baby-changing table for convenience. Such baby-changing tables typically include a table that may be pivoted between a closed position and an open position and also provide restraint means for securing the baby thereon. These types of tables are very convenient and provide a safe and secure baby-changing area. However, currently they do not include dispensable accessories associated therewith, such as a diaper, diaper wipes, lotion, baby powder, changing pad, or the like. It is therefore contended that there is a need to provide a vending machine therewith for dispensing such accessories.

Although the invention may be used in combination with a vending machine, the actual machine is not taught herein as such machines and their technology are very well known within the prior art and any type of suitable coin-operated dispensing machine may be used. Thus, the actual pre-packaged item is what we wish to address and resolve in a novel manner heretofore not taught within the known prior art.

Within the known prior art there have been many attempts to provide a diaper dispensing apparatus that may be coin-operated. Such attempts are taught within U.S. Pat. Nos. 5,678,727, 4,706,845, 4,574,822 and 3,970,217. Each of which provide diapers for dispensing but none provide a novel pre-packaged item including a diaper and associated accessories as taught herein as will be seen within the following specification.

### OBJECTS AND ADVANTAGES OF THE PRESENT INVENTION

It is therefore a primary object of the present invention to provide a pre-packaged dispensable item having novel features and includes a method of use. For example, in the preferred embodiment we provide an external wrap for sanitary containment of a disposable diaper positioned therein. The external wrap is not only functional for containment of the diaper but also functions as a sanitary placemat that is of a size and shape to fit appropriately upon the changing table. Also, most importantly the external wrap is in the form of a disposable bag for containment of a used soiled diaper and/or other disposable items associated therewith.

Other notable novel objects of the present invention may be to further include additional accessories of engineering choice. For example, positioned within the package there may be one or multiple packaged accessory items such as moist diaper wipes, lotion, DESITIN™, baby powder, etc.

Yet another object of the present invention is provide the noted external wrap so that when the diaper and/or accessories are positioned either thereon or therein they are contained in a sanitary manner that is most important. Furthermore, the external wrap when unfolded provides easy access of the items therein to the user. Thus, the manner in which the wrap is used is unique and provides unusual end results heretofore not taught. Also, the method of use is simple, less time consuming, and most efficient. Other objects and advantages will be seen when taken into consideration with the following drawings and specification.

### BRIEF DESCRIPTION OF THE DRAWINGS

FIG. 1 is substantially an overview of the preferred embodiment of the present invention when in the unfolded open position.

FIG. 2 is substantially an overview of the preferred embodiment of the present invention when in the folded closed position.

### DETAILED DESCRIPTION OF THE DRAWINGS

Referring now in detail to the drawings wherein like characters refer to like elements throughout the various views. It is to be understood the present invention is novel not because of the individual components respectively, but more so because of there use in combination that provides unusual results. For example, the actual components are pre-existing items and no modifications need be made thereto, as they are each fully functional as currently available, produced and sold. The actual components include at least a placemat (10) and a diaper (12). Other components are optional and may include packaged accessories of choice, such as a packaged moist diaper wipe (14) or packaged lotion (16) or the like. It is to be understood items (14) and (16) are only exemplary of some optional accessories and the invention is not to be limited thereto. Thus, numerous accessory items are possible and those actually used are dependant upon engineering, manufacturing choice and/or end user needs.

Referring now to the external wrapping represented by placemat (10) that is actually in the form of a disposable bag, respectively having an internal compartment (18). The external wrapping or placemat (10) is most important as it functions for multiple purposes. For example, the placemat (10) first provides a sanitary wrap for containment of diaper (12) and/or any other accessories positioned either thereon or therein. The placemat (10) or wrapping may be made from any suitable material of choice. However, even though plastic is an option, plastic alone may not be desirable as this may be dangerous and may not comply with safety codes. Therefore it is preferable that other materials, such as absorbent padded paper or the like are used for constructing placemat (10). Another option maybe to include an internal plastic coating (not shown) within internal compartment (18). This is simply to reduce undesirable odors from escaping there through, as odors from soiled diapers can be most unpleasant and may disturb others using the facility. Another option to resolve this matter would be to include a pleasant scent that may be impregnated within the material used for placemat (10).

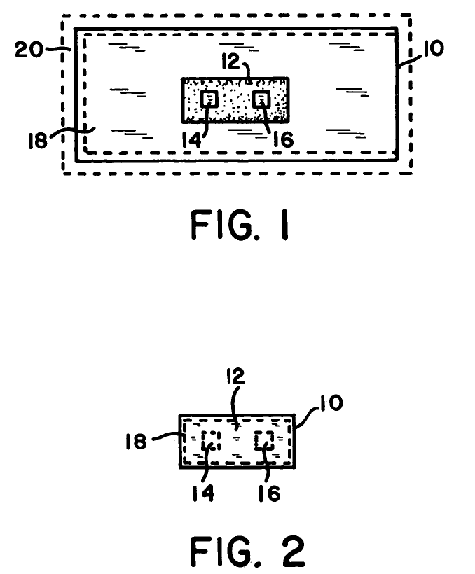

Furthermore placemat (10) is to be of a shape and size to suitably fit onto the pre-existing typical diaper-changing table (20) (illustrated in ghost lines for exemplary purposes) when in the unfolded open position, as depicted in FIG. 1. Thus, it is of a sufficient size to substantially cover the table (20) respectively, and provides a comfortable sanitary mat for placement of the baby when positioned thereon. Still a further option is to produce the placemat (10) in different colors of choice. For example, nowadays diapers may be differentiated by color when used by either girls or boys, such as blue for boys and pink for girls. Depending on the dispensing machine and/or engineering choices this option is exemplary and may be easily incorporated if so desired.

Referring now to diaper (12), which again can be any suitable pre-existing disposable diaper of choice. Furthermore, the invention is not to be limited to any specific size, shape, or brand, as different types of diapers may be offered and this is again dependant on the dispensing means of choice and/or the noted variable options.

Referring now to items (14) and (16), as previously addressed these are only exemplary of some of the optional features inherent within the invention. As presented herein (14) substantially represents a packaged moist diaper wipe and (16) substantially represents a package of lotion (16). Therefore any packaged item or substance of choice may be offered in combination with diaper (12).

We will now address the method of use for the present invention so as to further define the novel features associated therewith. A method of use for changing a babies diaper using a pre-packaged dispensable wrapped diaper with accessories comprising the following steps of:
a. locating a baby changing table, such as typically provided within most ladies restrooms;
b. positioning the baby changing table into an open position;
c. inserting a coin into a vending machine located in close proximity to the baby changing table, whereby automatically causing a pre-packaged wrapped diaper to be dispensed from the vending machine;
d. grasping the now dispensed pre-packaged wrapped diaper;
e. unfolding the external wrapping in a manner that exposes the sanitary side of the external wrapping and the diaper and any accessories located therein;
f. grasping the diaper and/or any accessories;
g. positioning the external wrapping onto the baby changing table with the sanitary side facing upward;
h. positioning the baby onto the sanitary side of the external wrapping;
i. securing the baby thereon with restraining straps located on the baby changing table;
j. removing the soiled diaper from the baby in the usual manner;
k. cleaning the baby with the noted accessories;
l. positioning the newly dispensed diaper onto the baby in the usual manner and securing in place;
m. removing the restraining straps, the baby from the external wrapping and the baby changing table;
n. grasping the soiled diaper along with any packages associated with the accessories;
o. grasping the external wrapping which is actually in the form of a disposable bag;
p. opening the disposable bag;
q. inserting the soiled diaper along with any packages associated with the accessories into the disposable bag; and;
r. disposing of the disposable bag and its contents into a typical trash receptacle in the usual manner.

It can now be seen we have herein provided a new and novel means for changing a babies diaper using a pre-packaged dispensable diaper and accessories associated therewith. The pre-packaged diaper and accessories being contained within a sanitary external wrapping that is actually in the form of a disposable bag. Thus, the disposable bag, the diaper and/or accessories in combination provide unusual results heretofore not taught.

Although the invention has been herein shown and described in what is conceived to be the most practical and preferred embodiment, it is recognized that departures may be made there from within the scope and spirit of the invention, which is not to be limited to the details disclosed herein but is to be accorded the full scope of the claims so as to embrace any and all equivalent devices and apparatuses.

Having described our invention, what we claim as new and desire to secure by Letters Patent is:

1. A pre-packaged dispensable diaper in combination comprising: an external wrapping; and a disposable diaper; said external wrapping having a folded closed position and an unfolded open position, said external wrapping when in said unfolded open position further functions as a sanitary placemat; said external wrapping when in said folded closed position provides a sanitary wrap for containment of said diaper therein, and said external wrapping being in the form of a disposable bag.

2. The pre-packaged dispensable diaper of claim 1 wherein said external wrapping when in said folded closed position provides a sanitary wrap for containment of said diaper and at least one accessory packaged item therein.

3. The pre-packaged dispensable diaper of claim 2 wherein said at least one accessory packaged item is a moist diaper wipe.

4. The pre-packaged dispensable diaper of claim 2 wherein said at least one accessory packaged item is lotion.

5. The pre-packaged dispensable diaper of claim 1 wherein said external wrapping is impregnated with a pleasant scent.

6. A method of use for changing a babies diaper using a pre-packaged dispensable wrapped diaper with accessories comprising the following steps of:
a. locating a baby changing table, such as typically provided within most ladies restrooms;
b. positioning said baby changing table into an open position;
c. inserting a coin into a vending machine located in close proximity to said baby changing table, whereby automatically causing said pre-packaged wrapped diaper to be dispensed from said vending machine;
d. grasping the now dispensed said pre-packaged wrapped diaper;
e. unfolding external wrapping in a manner that exposes a sanitary side of said external wrapping and said diaper and said accessories located therein;
f. grasping said diaper and/or said accessories;
g. positioning said external wrapping onto said baby changing table with said sanitary side facing upward;
h. positioning said baby onto said sanitary side of said external wrapping;
i. securing said baby thereon with restraining straps located on said baby changing table;
j. removing said soiled diaper from said baby in the usual manner;
k. cleaning said baby with the noted accessories;
l. positioning said diaper onto said baby in the usual manner and securing in place;
m. removing said restraining straps, said baby from said external wrapping and said baby changing table;
n. grasping said soiled diaper along with any packages associated with said accessories;
o. grasping said external wrapping which is actually in the form of a disposable bag;
p. opening said disposable bag;
q. inserting said soiled diaper along with said packages associated with said accessories into said disposable bag; and;
r. disposing of said disposable bag and its contents into a typical trash receptacle in the usual manner.
